(19) United States
(12) Patent Application Publication
(10) Pub. No.: US 2005/0004419 A1
(43) Pub. Date: Jan. 6, 2005

(54) Hydraulic assist method and system

(76) Inventors: Jacob Lavee, Ramat Gan (IL); Menashe Shahar, Korazim Drom Hagolan (IL); Zvi Ben Shalom, Yavne (IL)

Correspondence Address: NATH & ASSOCIATES PLLC, Sixth Floor, 1030 15th Street, N.W., Washington, DC, 20005, US

(21) Appl. No.: 10/612,337

(22) Filed: Jul. 3, 2003

(51) Int. Cl. A61N 1/362

(52) U.S. Cl. 600/16

(57) ABSTRACT

A hydraulic system and method for supporting a body organ, the system comprising a closed loop liquid-tight tubing fitted with a pressure generator for propelling a liquid through the system, an organ engaging member connected to a pressure chamber via a discharge valve for controlled discharge of liquid into the organ inflatable pressure member. The organ engaging member comprises an inflatable pressure member suited for receiving the organ. There is further provided at least one control valve for selectively controlling liquid flow through the system and a controller for selectively controlling the discharge valve and the at least one control valve.

### FIELD OF THE INVENTION

The present invention relates to a hydraulic method and a system utilizing the system useful for various therapeutic purposes associated with various body organs such as, cardiac assist, limb massaging, etc.

### BACKGROUND OF THE INVENTION

It is often required to assist a failing body organ in, performing its normal sequential operation. Examples of such instances are:

I. A failing heart where cardiac assistance is necessary in case a patients cardiac output drops below the blood supply required to sustain proper blood perfusion. In such a case a ventricular-assist-device (VAD) is used to bridge a patient until his natural heart recovers, or until a suitable donor heart is found for heart transplantation or as a permanent alternative to heart transplantation. A VAD is useful in complementing or taking over the pumping function of one or both sides of a failing heart by which they unload the assisted ventricle. Several VAD devices and method are known, for example, those disclosed in, U.S. Pat. Nos. 4,192,293, 5,131,905, 5,429,584, 5,406,422, 5,713,954, 5,800,525, 5,971,910, and 6,508,756.

II. Massaging a limb, e.g. a patient's leg, in case of thrombosis or other vein diseases which are very common among diabetic patients. Massaging the blood vessels, either directly or by applying massaging motion on the limb may, in many cases, save the limb and prevent it from being amputated.

It is an object of the present invention to provide a hydraulic system and a method utilizing said system for various therapeutic purposes associated with various body organs to thereby stimulate their motion. SUMMARY OF THE. INVENTION

According to the present invention there is provided hydraulic system for supporting a body organ., the system comprising a closed loop liquid-tight tubing fitted with a pressure generator for propelling a liquid through the system, an organ engaging member connected to a pressure chamber via a discharge valve for controlled discharge of liquid into the organ engaging member; said organ engaging member comprising an inflatable pressure member suited for receiving the organ; at least one control valve for selectively controlling liquid flow through the system; and a controller for selectively controlling the discharge valve and the at least one control valve.

According to another aspect of the invention there is provided a method for stimulating motion to a body organ, the method comprising the following steps:
(a) obtaining body-organ hydraulic assist system comprising a pressure generator for propelling a liquid through the system, a pressure chamber, an organ engaging member connected to the pressure chamber and fitted with an inflatable pressure member, valves for selectively controlling liquid flow through the system and for controlled discharge of liquid to the organ engaging member; a controller for selectively controlling the valve means;
(b) fitting the inflatable pressure member over the body-organ; and
(c) activating the pressure generator and the controller.

A principal operating sequence of the system according to the invention comprise charging the pressure chamber, inflating the inflatable pressure member of the organ engaging member to thereby apply a desired pressure on the organ. Thereafter the pressure member is deflated and the system is ready for a new operating cycle, depending on a particular control algorithm.

The valves are controlled by the controller at a discrete order corresponding with an algorithm suitable for a therapeutic routine and preferably the controller generates control signals responsive to signals corresponding with physiologicafly-related parameters obtained from the body.

According to a particular embodiment the hydraulic assist method comprises a first one-way valve fitted between the pressure generator and the pressure chamber, a second one-way valve fitted between the organ engaging member and the pressure generator, a pressure discharge valve for controlled discharge of liquid from the pressure chamber into the organ engaging member, and a circulation one-way valve mounted in parallel relation to the pressure generator, on a tube segment extending between the pressure generator and the first one-way valve, and between the second one-way valve and the pressure generator.

According to one embodiment, the method comprises two principal control stages, namely;
(a) a charging stage wherein the pressure discharge valve and the circulating valve are closed, the first valve and the second valve are open, and the pressure generator is active;
(b) a pressure stage wherein the discharge valve is open to thereby facilitate inflating the pressure member, and the circulating valve is open while the pressure generator is active; whilst the first valve and the second valve are closed.

It is however appreciated that a variety of control algorithms are possible for obtaining a sequence of a work cycle as above.

Where the body organ is a heart and the system operates as a cardiac assist system, it is desired that the system operates in cadence with the normal functioning of the heart. In particular, the pressure chamber is charged with pressurized liquid during the diastolic heart period and the organ engaging member is inflated during the systolic period.

The device according to the present invention may be suited for entirely implanting or partially implanting, where at least the organ engaging member is transplanted and other components are externally retained.

### BRIEF DESCRIPTION OF THE DRAWINGS

In order to understand the invention and to see how it may be carried out in practice, some embodiments will now be described, by way of non-limiting examples only, with reference to the accompanying drawings, in which:

FIG. 1 is a schematic representation of a system according to the present invention;

FIG. 2 is a time/volume graphic representation of a cycle of a failing heart superimposed with the performance with a system according to the present invention;

FIGS. 3A and 3B are schematic representations of the principal control stages of a system according to an embodiment of the present invention; and

FIGS. 4A to 4E are schematic representations of a specific control algorithm of a system according to the present invention.

### DETAILED DESCRIPTION OF THE INVENTION

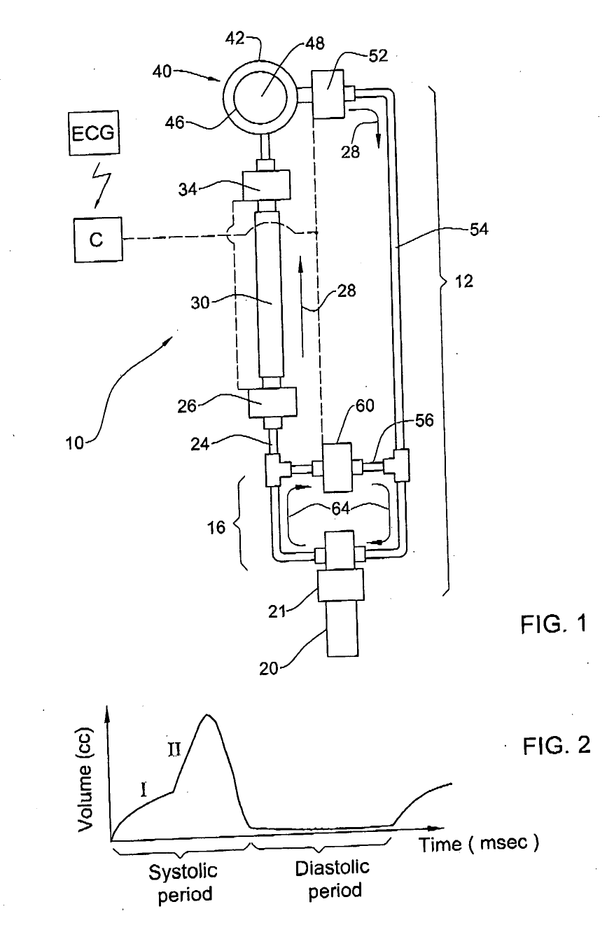

Attention is first directed to FIG. 1 of the drawings illustrating a system in accordance with the present invention, generally designated 10 which will be hereinafter exemplified in connection with a cardiac-assist-device (CAD) and comprising a primary pipe circuit generally designated 12 and a secondary pipe circuit generally designated 16. The primary pipe circuit comprises a pressure generator 20 which is a hydraulic pump designed to operate at low flow rates and suited for continuous and long lasting operation. Pressure generator 20 is fitted with a power source e.g. battery 21. However, it should be appreciated that other forms of power source may be used, such as, an external battery (where power is supplied by suitable conductive means) or an internal battery chargeable by externally applied induction, etc.

Preferably, a non-expandable/non-inflatable pipe segment 24 communicates between the pressure generator 20 and a first valve 26 which is a one-way valve admitting flow only in a flow direction illustrated by arrows 28. Valve 26, like all other valves in the system is controlled by a controller C communicating therewith by means of control lines illustrated in FIG. 1 by dashed lines.

First valve 26 is in flow communication with a pressure chamber 30 which is a pressurized liquid accumulator, for example, made of a container having considerably elastic walls, e.g. silicon or a metallic membrane deformable between a constricted position and an expanded, pressurized position.

Downstream of the pressure chamber 30 there is provided a discharge valve designated 34 which is also a one-way valve permitting flow only in the direction of arrows 28 and which is also controlled and governed by controller C as will be apparent hereinafter.

An organ engaging member 40 comprises a casing 42 made of a rigid, non-deformable material and accommodating an inflatable pressure member 46 being in flow communication with the discharge valve 34. The inflatable pressure member 46 is made of a pliable/flexible material and it is not necessary for the inflatable pressure member to be elastic. The inner shape of the inflatable pressure member corresponds with the shape of the concerned organ. Inflating the pressure member 46 entails inward radial constriction of space 48.

The inflatable pressure member 46 is further in flow communication with a second valve 52 which is a one-way discharge valve also controllable by controllable C and which is in flow communication via a return pipe segment 54 with the pressure generator 20.

The secondary pipe circuit 16 comprises a pipe segment 56 extending between pipe segments 24 and 54 with a circulating valve 60 mounted thereon and controllable by controller C. Circulating valve 60 is a one-way valve allowing flow only in the direction of arrows 64, i.e. in collaboration with the flow through the primary pipe circuit 12.

Typically, all the components of the system are biologically acceptable and are made of approved material. Furthermore, the piping segments are preferably made of essentially non-elastic material to thereby prevent undesired pressure loss at some regions of the system and to prevent wearing of the mechanical components.

The liquid flowing through the system is a physiological acceptable solution so that in case of leakage no harm is caused.

In practice, the device may be completely implanted into a body, e.g. in case of a cardiac assist device wherein the heart is cradled within the organ engaging member. However, at times, and depending on the organ concerned, it may be appropriate that some of the components of the system, e.g. pressure generator 20 with its power supply 21 and controller C be externally attached for maintenance and performing different functions.

The controller C may be a programmable controller and may even be associated with an. ECG device so as to change the performance of the system depending on physiological parameters of the patient.

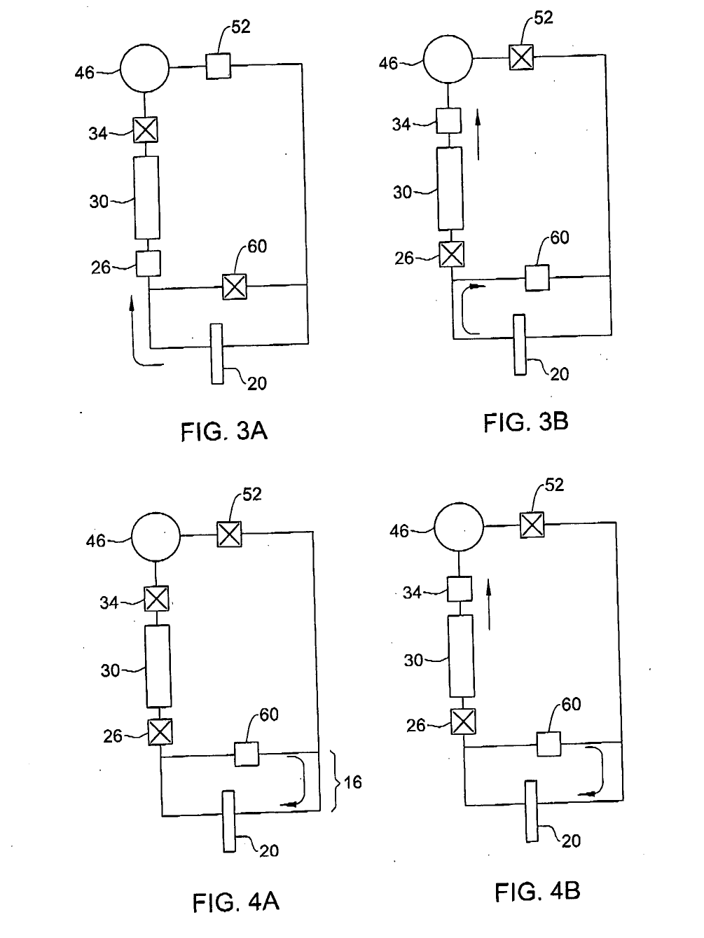

Further reference is directed also to FIGS. 3A and 3B wherein like elements as in FIG. 1 were given the same reference numbers. A work cycle comprises a sequence of two principal stages, namely a charging stage (FIG. 3A) and a pressure stage (FIG. 3B).

At the charging stage of FIG. 3A the inflatable pressure chamber 46 is drained (by opening second valve 52) and discharge valve 34 and the circulating valve 60 are closed and the first valve 26 is open, such that pressure generated by pressure generator 20 is charged into the pressure chamber 30 which act as an accumulator. Upon completing the charging stage, the controller C (FIG. 1) switches the system into to the pressure stage as in FIG. 3B. In this stage the second valve 52 and the first valve 26 are closed, whilst the discharge valve 34 is opened so as to facilitate discharging the pressure chamber 30 and inflating the inflatable pressure member 46 of the organ engaging member 40. Further at this stage, the circulating valve 60 opens, so as to circulate the working liquid through the secondary pipe circuit 16 (FIG. 1) whilst the pressure generator 20 continues to work.

The two principal stages of FIGS. 3A and 3B are continuously repeated in cadence with the self pace of the heart. Accordingly, the charging stage (FIG. 3A) will typically occur during a diastolic heart period (FIG. 2), and the pressure stage (FIG. 3B) will occur during the self systolic operation of the heart (corresponding with lines I in FIG. 2).

It is preferable that the system is continuously controlled and governed by physiological parameters in accordance with the physiological condition of the heart, as mentioned hereinabove and as known in the art.

Whilst the systems has been described and disclosed with reference to a cardiac device, it should be obvious to a skilled person that the system may also be used for assisting other organs, e.g. massaging blood vessels in limbs, etc.

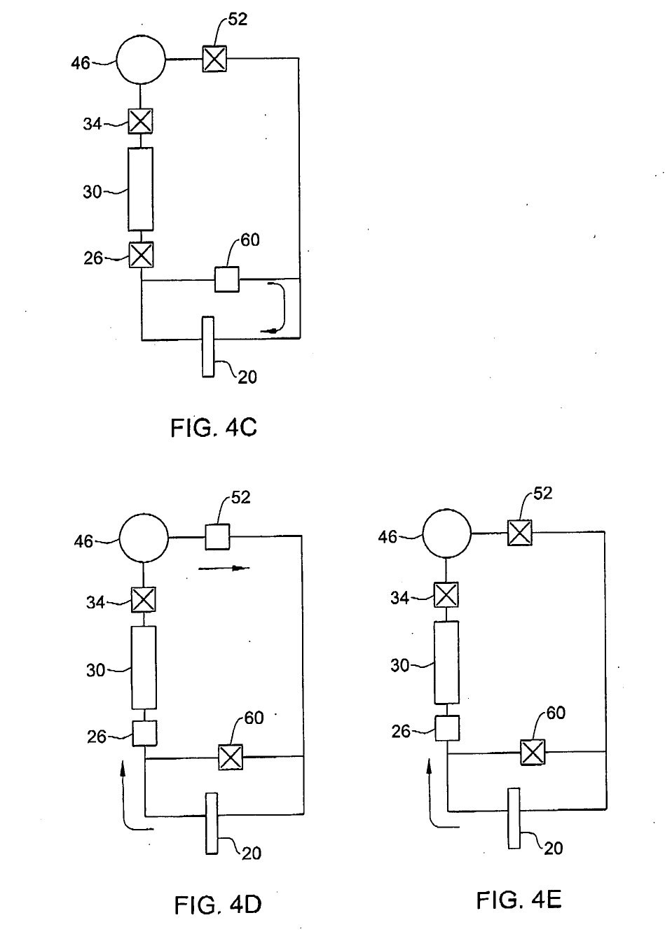

A somewhat different control algorithm is illustrated in FIGS. 4A to 4E wherein for sake of clarity like elements as in FIGS. 1 and 3A and 313 were given the same reference numbers.

At the initial stage of FIG. 4A inflatable pressure member 46 is deflated (un-pressurized) whilst the pressure chamber 30 is charged (under pressure). The valves 26, 34 and 52 are in closed position and circulation valve 60 is open to thereby facilitate circulation in secondary pipe circuit 16 by means of pressure generator (pump) 20. At the next step (FIG. 4B), discharge valve 34 is opened whereby liquid. After the pressure stage of FIG. 4B, discharge valve 34 closes again (FIG. 4C). Then, valve 52 opens to drain (deflate) the pressure member 46, valve 26 opens and valve 60 closes whereby liquid now flows to the pressure chamber 30 (FIG. 4D). Finally, valve 52 closes (FIG. 4E) and upon completing charge of the pressure chamber 30, the system is now ready for a new sequence beginning at FIG. 4A.

In order to avoid excessive heat built-up and excessive wear of the system, it is desirable that either the first valve or the circulation valve be open, while the pump/pressure generator is active. A possible alternative may be halting pressure generation while both said valves are closed.

Whilst two particular examples of control algorithms have been disclosed herein above, it is to be appreciated that other control algorithms may be utilized to obtained a controlled sequence of operation according to various operating patterns. For example, valves may be opened/closed simultaneously rather than in series, etc.

Furthermore, it is apparent that other modifications of the device are possible, such as use of other forms of power source, etc. Evermore so, the device may be suited for entirely implanting or partially implanting, where at least the organ engaging member is transplanted and other components are externally retained.

1. A hydraulic system for supporting a body organ, the system comprising a closed loop liquid-tight tubing fitted with a pressure generator for propelling a liquid through the system, an organ engaging member connected to a pressure chamber via a discharge valve for controlled discharge of liquid into the organ inflatable pressure member; said organ engaging member comprising an inflatable pressure member suited for receiving the organ; at least one control valve for selectively controlling liquid flow through the system; and a controller for selectively controlling the discharge valve and the at least one control valve.

2. A system according to claim 1, wherein the organ engaging member comprises a rigid housing accommodating the inflatable pressure member, where the organ is received within a space formed by the inflatable member and whereby inflating the inflatable member entails constriction of the space.

3. A system according to claim 2, wherein the inflatable member is annular.

4. A system according to claim 1, wherein the liquid flowing through the system, is a physiological acceptable solution.

5. A system according to claim 1, wherein the pressure chamber is a pressure accumulator.

6. A system according to claim 1, wherein the pressure chamber comprises an accumulator fitted with an elastic membrane.

7. A system according to claim 1., wherein the pressure discharge valve and control valves are one-way valves.

8. A system according to claim 1, wherein the controller is programmable.

9. A system according to claim 1, wherein the controller generates control signals responsive to signals corresponding with physiologically-related parameters obtained from the body.

10. A system according to claim 1, being a cardiac assist device and where the organ engaging member is fitted around the heart.

11. A system according to claim 10, wherein the controller generates control signals responsive to heart condition signals received from an associated ECG device.

12. A system according to claim 10, wherein the wherein the discharge valve is activated to exercise the organ engaging member at a rate corresponding with the self systolic pace of the heart.

13. A system according to claim 1, wherein the where the organ is a limb and where the system is used for messaging the limb and stimulating blood flow there through.

14. A system according to claim 1, wherein the used as a peristaltic assist device.

15. A system according to claim 1, wherein the where the pressure generator is a hydraulic pump.

16. A system according to claim 15, wherein the where the pump is a low flow-rate pump.

17. A system according to claim 16, wherein the pump continuously operates.

18. A system according to claim. 1, wherein the organ engaging member is inflated to an extent below its elastic deformation.

19. A system according to claim 1, wherein the closed loop liquid-tight tubing comprises non-elastic components.

20. A system according to claim 1, wherein a first one-way valve is fitted between the pressure generator and the pressure chamber

21. A system according to claim 20 wherein a second one-way valve fitted between the organ engaging member and the pressure generator.

22. A system according to claim 21, further comprising a circulation one-way valve mounted in parallel relation to the pressure generator, on a tube segment extending between the pressure generator and the first one-way valve, and between the second one-way valve and the pressure generator.

23. A body-organ hydraulic assist system comprising a pressure generator for propelling a liquid through the system, a pressure chamber, an organ engaging member connected to the pressure chamber and fitted with an inflatable pressure member, valve means for selectively controlling liquid flow through the system and for controlled discharge of liquid to the organ engaging member; a controller for selectively controlling the valve means.

24. A system comprising according to claim 1, comprising two or more organ engaging members, associated with corresponding pressure chambers with respective valves, and at least one pressure generator for propelling a liquid through the system; said controller discreetly controlling the system so as to obtain a sequential performance of the organ engaging members, in a peristaltic manner.

25. A hydraulic assist system for stimulating motion to a body organ, the system comprising a primary pipe circuit being in flow communication with a secondary pipe circuit; said primary pipe circuit comprising an organ engaging member fitted with a hydraulically inflatable pressure member adapted for receiving said organ, a pressure chamber coupled to a pressure generator via a pressure discharge valve; the system further comprising control valves for selectively circulating liquid through the primary pipe circuit or through the secondary pipe circuit, and a controller for controlling said discharge valve and said valves.

26. A system according to claim 25, wherein the primary pipe circuit comprises a first one-way valve fitted between the pressure generator and the pressure chamber, a second one-way valve fitted between the organ engaging member and the pressure generator.

27. A system according to claim 26, wherein the secondary pipe circuit comprises a circulation one-way valve mounted in parallel relation to the pressure generator, on a pipe segment extending between the pressure generator and the first one-way valve, and between the second one-way valve and the pressure generator.

28. A method for stimulating motion to a body organ, the method comprising the following steps:
(a) obtaining body-organ hydraulic assist system comprising a pressure generator for propelling a liquid through the system, a pressure chamber, an organ engaging member connected to the pressure chamber and fitted with an inflatable pressure member, valves for selectively controlling liquid flow through the system and for controlled discharge of liquid to the organ engaging member; a controller for selectively controlling the valve means;
(b) fitting the inflatable pressure member over the limb;
(c) activating the pressure generator and the controller.

29. A method according to claim 28 for assisting a failing ventricular system, the method further comprising the steps of:
(d) obtaining condition signals indicative to a heart condition, obtained from an associated ECG device;
(e) said controller generating control signals to the valves of the system, responsive to said condition signals.

30. A method according to claim 29, wherein the controller generates the control signals in cadence with the normal functioning of the heart.

31. A method according to claim 29, wherein the pressure chamber is charged with liquid during the diastolic heart period.

32. A method according to claim 28, wherein the hydraulic assist system comprises a first one-way valve fitted between the pressure generator and the pressure chamber, a second one-way valve fitted between the organ engaging member and the pressure generator, a pressure discharge valve for controlled discharge of liquid from the pressure chamber into the organ engaging member, and a circulation one-way valve mounted in parallel relation to the pressure generator, on a tube segment extending between the pressure generator and the first one-way valve, and between the second one-way valve and the pressure generator.

33. A method according to claim 32 comprising two principal control stages;
I. a charging stage wherein the pressure discharge valve and the circulating valve are closed, the first valve and the second valve are open., and the pressure generator is active; and
II. a pressure stage wherein the discharge valve is open to thereby facilitate inflating the pressure member, and the circulating valve is open where the pressure generator is active; whilst the first valve and the second valve are closed.

34. A method according to claim 32, comprising the following steps:
(a) closing the pressure discharge valve and the circulating valve;
(b) opening the second valve so as to drain the organ engaging member;
(c) opening the first valve and charging the pressure chamber;
(d) closing the first valve and the second valve and simultaneously opening the circulation valve;
(e) controllably opening the pressure discharge valve so as to inflate the pressure member;
(f) repeating steps (a) to (e).

35. A method according to claim 34, wherein the system is used as a ventricular assist device, wherein step (e) is carried out at the beginning of a systolic heart period.

36. A method according to claim 34, wherein steps (c-d) take place during the diastolic heart period.

37. A method according to claim 34, wherein the operational steps take place in cadence with the normal functioning of the heart.

38. A method according to claim 34, wherein the pressure generator continuously operates.

39. A method according to claim 28, wherein the hydraulic assist system comprises a first one-way valve fitted between the pressure generator and the pressure chamber, a second one-way valve fitted between the organ engaging member and the pressure generator, a pressure discharge valve for controlled discharge of liquid from the pressure chamber into the organ engaging member, and a circulation one-way valve mounted in parallel relation to the pressure generator, on a tube segment extending between the pressure generator and the first one-way valve, and between the second one-way valve and the pressure generator; the method comprises the following control steps;
(a) after charging the pressure chamber, closing the first valve, the second valve, and opening the circulating valve;
(b) opening the discharge valve to pressurize the wherein the hydraulic assist system comprises a first one-way valve fitted between the pressure generator and the pressure chamber, a second one-way valve fitted between the organ engaging member and the pressure generator, a pressure discharge valve for controlled discharge of liquid from the pressure chamber into the organ engaging member, and a circulation one-way valve mounted in parallel relation to the pressure generator, on a tube segment extending between the pressure generator and the first one-way valve, and between the second one-way valve and the pressure generator; the method comprises the following control steps;
(c) closing the discharge valve;
(d) opening the second valve to discharge the inflatable pressure member;
(e) opening the first valve and closing the circulating valve; and
(f) repeating steps (a) to (e).

40. A method according to claim 39, wherein the pressure generator continuously operates.
